(19) United States
(12) Patent Application Publication
(10) Pub. No.: US 2012/0158031 A1
(43) Pub. Date: Jun. 21, 2012

(54) Scleral marker for vitreoretinal entry system

(76) Inventor: Matthew D. McCawley, San Clemente, CA (US)

(21) Appl. No.: 12/974,352

(22) Filed: Dec. 21, 2010

(51) Int. Cl. A61B 17/34; A61F 9/007

(52) U.S. Cl. 606/185

(57) ABSTRACT

A surgical trocar assembly with a marker element is disclosed. The assembly comprises a trocar, a handle, and a marker element. The trocar is constructed as an elongated member defined by distal and proximal ends. The distal end further includes a tissue piercing tip. The marker element defines a shoulder and includes at least one contacting member that extends in a distal direction from the shoulder. The contacting member is configured to contact a surface of tissue adjacent an incision site created by the tissue piercing tip so as to provide a visual indication of the incision site when the trocar is removed from the incision site.

### TECHNICAL FIELD

The present disclosure relates generally to micro-incision surgical trocars, and more particularly to a method and apparatus for marking a surgical incision site.

### BACKGROUND

Micro-incision vitrectomy surgical procedures frequently utilize a trocar cannula to provide an entry port into the posterior segment of the eye. More specifically, in a typical procedure, the trocar cannula is inserted through the wall of the eye, typically at the pars plana, and deforms the sclera to create a rigid port to permit multiple instrument exchanges during a vitreoretinal surgery. The trocar cannula remains inserted in the sclera throughout the entire process.

For surgical procedures that do not require numerous instrument exchanges, it is beneficial to eliminate the cannula, thereby significantly reducing the wound size necessitated by the surgical procedure. However, a disadvantage of eliminating a trocar cannula is the inability to find the small incisions created by the micro-incision vitrectomy instruments. Indeed, as micro-incision vitrectomy technology moves to less invasive, and smaller sized instruments (i.e. 27 gauge), the elimination of a trocar cannula makes it increasingly difficult to see and find the incision location when instruments are introduced into the posterior chamber. Moreover, the incision sights may also be obscured by the fluids and mucosa that naturally coat the surface of the eye. Accordingly, the inability to quickly locate the incisions may increase the duration of the surgical procedures, result in incorrect placement of the surgical instruments or require a creation of a new surgical incision, thereby resulting in more trauma to the patient. Accordingly, what is needed is a mechanism that temporarily marks the incision site to allow the surgeon to quickly locate the incision site during surgery.

### BRIEF SUMMARY

A surgical trocar assembly with a marker element is disclosed. The assembly comprises a trocar, a handle, and a marker element. The trocar is constructed as an elongated member defined by distal and proximal ends. The distal end further includes a tissue piercing tip. The marker element defines a shoulder and includes at least one contacting member that extends in a distal direction from the shoulder. The contacting member is configured to contact a surface of tissue adjacent an incision site created by the tissue piercing tip so as to provide a visual indication of the incision site when the trocar is removed from the incision site.

### BRIEF DESCRIPTION OF THE DRAWINGS

Exemplary embodiments of the present disclosure will now by described by way of example in greater detail with reference to the attached figures, in which:

FIG. 1A is a partial cross-sectional perspective view of a surgical trocar with an integrated marking element attached thereto;

FIG. 1B is an enlarged view of the marking element shown in circled area 1B taken from FIG. 1A;

FIG. 2A is a partial cross-sectional view of the surgical trocar as it is being inserted into an eye;

FIG. 2B is an enlarged view of the marking element shown in circled area 2B taken from FIG. 2A;

FIG. 3 is a perspective view of an eye, illustrating a marked incision site created by the marking element of the surgical trocar of FIG. 1A.

FIG. 4A is a perspective view of a surgical trocar with an alternative marking element attached thereto;

FIG. 4B is an enlarged view of the marking element shown in circled area 4A taken from FIG. 4A;

FIG. 4C is an end view of the marker element shown in FIG. 4B.

FIG. 5 is a perspective view of an eye, illustrating a marked incision site created by the marking element of the surgical trocar of FIG. 4A.

FIG. 6A is a perspective view of a surgical trocar with an alternative marking element attached thereto;

FIG. 6B is an enlarged view of the marking element shown in circled area 6A taken from FIG. 6A;

FIG. 6C is an end view of the marker element shown in FIG. 6B.

FIG. 7 is a perspective view of an eye, illustrating a marked incision site created by the marker element of the surgical trocar of FIG. 6A.

### DETAILED DESCRIPTION OF THE INVENTION

Referring now to the discussion that follows and also to the drawings, illustrative approaches to the disclosed devices and methods are shown in detail. Although the drawings represent some possible approaches, the drawings are not necessarily to scale and certain features may be exaggerated, removed, or partially sectioned to better illustrate and explain the present disclosure. Further the descriptions set forth herein are not intended to be exhaustive or otherwise limit or restrict the claims to the precise forms and configurations shown in the drawings and disclosed in the following detailed description.

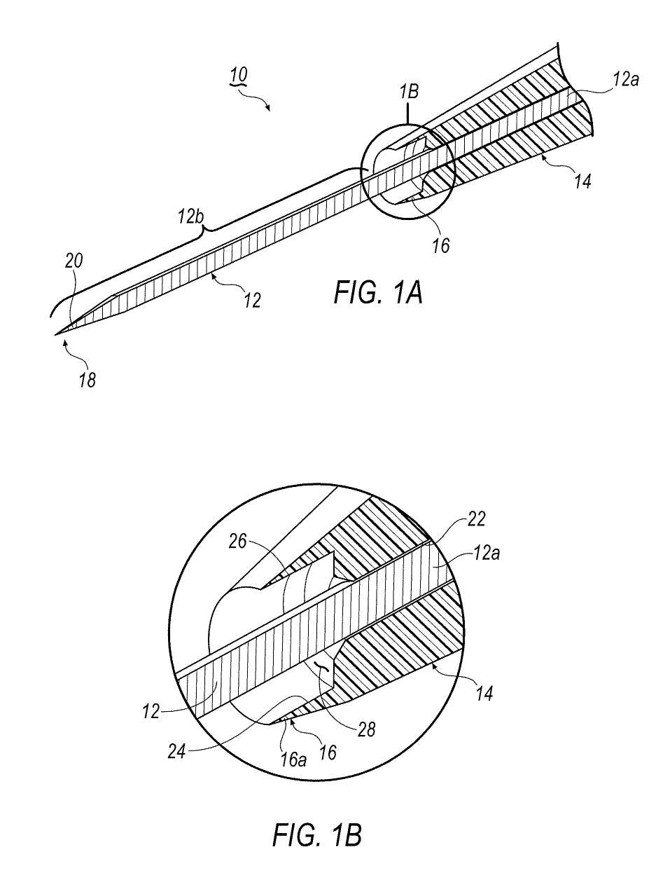

Referring to FIGS. 1A-3, a first exemplary arrangement of a surgical trocar assembly 10 will be discussed. Surgical trocar assembly 10 comprises a trocar 12, a handle 14, and a marker element 16. Trocar 12 is generally constructed as an elongated stylet and defined by a distal end 18. Distal end 18 is configured with a tissue piercing tip 20 to facilitate penetration of trocar 12 into a patient's tissue, such as, for example, the eye.

In one exemplary arrangement, handle 14 includes a mounting channel 22 (best seen in FIG. 1B) that receives a proximal portion 12a of trocar 12. Proximal portion 12a of trocar 12 is fixedly secured in channel 22. More specifically, proximal portion 12a is inserted within handle 14 a predetermined distance, such that a working portion 12b of trocar 12 extends distally away from handle 14 by a predetermined distance that is configured to reach a desired location within a patient.

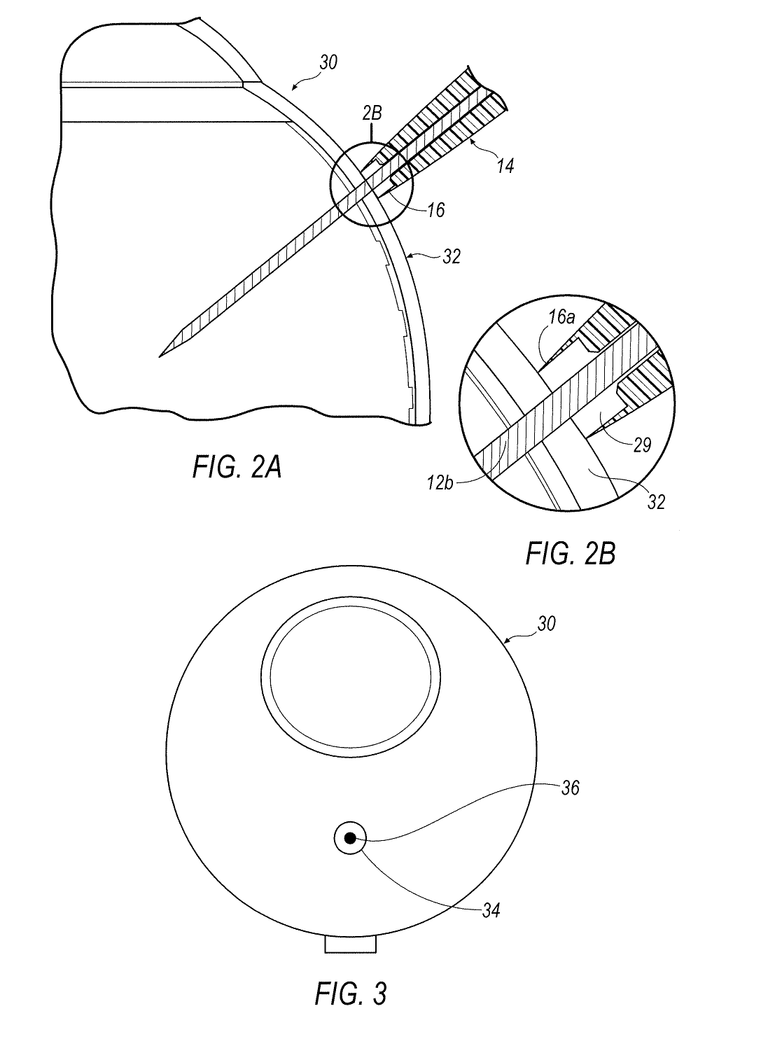

Marker element 16 is connected to handle 14. In one exemplary configuration, marker element 16 is configured as an annular member defined by an internal surface 24 and an external surface 26 to cooperate to form a generally circular and tapered marker such that marker element 16 is arranged concentrically around trocar 12. More specifically, tapered portion 16a extends distally outward from a distal shoulder 28 of handle 14. This configuration forms a recess 29 (best seen in FIG. 2B) between marker element 16 and trocar 12. In one exemplary arrangement, tapered portion 16a may further be configured as an ink-filled felt tip. In an alternative arrangement, tapered portion 16a may also employ a bioreactive stain or dye.

Referring now to FIGS. 2A-3, use of surgical trocar assembly 10 will now be explained in the context of a sclerotomy. However, it is understood that surgical trocar assembly 10 may be used in other surgical applications, particularly those which use micro-surgical instruments.

In operation, once a surgical site has been selected, tissue piercing tip 20 is inserted through the sclera 32, the fibrous membrane that cooperates with the cornea to form the outer covering and protection of eyeball 30. More specifically, working portion 12b of trocar 12 is inserted into eyeball 30, a predetermined distance until tapered portion 16a contacts sclera 32. This contact deposits a temporary, visible ring 34 around the incision site 36 created by trocar 12. To accommodate varying depths of insertion, recess 29 permits tapered portions 16a to slightly deform and press against sclera 32.

When trocar 12 is removed from incision site 36, ring 34 remains visible for a predetermined time period, such as the length of time for a surgical procedure. Accordingly, during a surgical procedure, various instruments may be selectively inserted within incision site 36 quickly and easily as incision site 36 is visible to the surgeon.

In an alternative arrangement, tapered portions 16a may not be ink-filled, but rather, tapered portions 16a may be pressed against sclera 32 so as to form a temporary, but visible impression 34 surrounding incision site 36 created by trocar 12. After a predetermined time period, impression 34 will disappear.

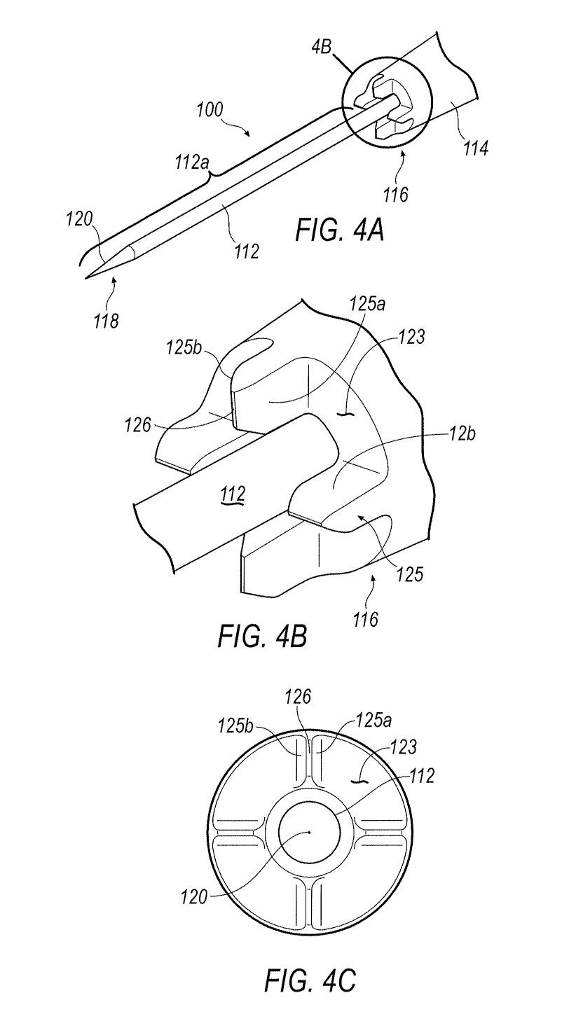

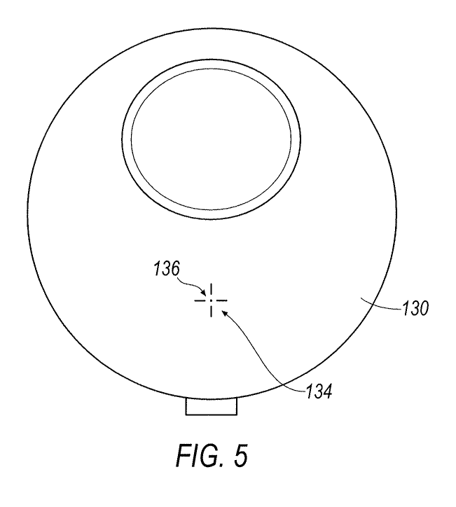

Referring to FIGS. 4A-5, an alternative arrangement of a surgical trocar assembly 100 will now be discussed. Surgical trocar assembly 100 comprises a trocar 112, a handle 114, and a marker element 116. Trocar 112 is generally constructed as an elongated stylet and defined by a distal end 118. Distal end 118 is configured with a tissue piercing tip 120 to facilitate penetration of trocar 112 into a patient's tissue, such as, for example, the eye.

In one exemplary arrangement, handle 114 is configured with a mounting channel (not shown) that receives a proximal portion of trocar 112. The proximal portion of trocar 112 is fixedly secured within the channel. More specifically, the proximal portion of trocar 112 is inserted within handle 114 by a predetermined distance, such that a working portion 112a of trocar 112 extends distally away from handle 114 by a predetermined distance that is configured to reach a desired location within a patient.

Marker element 116 is connected to handle 114. In one exemplary configuration, marker element 116 is configured as a generally annular member positioned around trocar 112, and defined by one or more contacting members 125 that extend in a distal direction from a shoulder 123 of handle 114. In one exemplary arrangement, for example, as shown in FIG. 4B, there are four contacting members 125, arranged equi-distance from one another. Each contacting member 125 is defined by opposing surfaces 125a, 125b with a distally oriented land member 126 positioned therebetween. In one exemplary arrangement, opposing surfaces 125a, 125b are contoured inwardly in the distal direction such that a width of each land member 126 may be relatively small. In another exemplary arrangement, each land member 126 may be oriented so as to be transverse to an axis extending through trocar 112. However, it is understood that opposing surfaces 125a, 125b need not be contoured at all, and other sizes and shapes of land members 126 may be employed. Further, any number of contacting members 125 may be employed.

Contacting members 125 may be integrally formed with handle 114, as shown in FIG. 4A. Alternatively, marker element 116 may be a separate unit that may be selectively attached to a distal end of handle 114 (not shown). For example, marker element 116 may be formed with a recess and an trocar opening, whereby the recess is configured to receive the distal end of handle 114 in a friction fit arrangement when marker element 116 is slid onto trocar 112 via the trocar opening.

Referring now to FIG. 5, use of surgical trocar assembly 100 will now be explained in the context of a sclerotomy. However, it is understood that surgical trocar assembly 100 may be used in other surgical applications, particularly those which use micro-surgical instruments.

In operation, once a surgical site has been selected, tissue piercing tip 120 is inserted through the sclera of eyeball 130. More specifically, working portion 112a of trocar 112 is inserted into eyeball 130, a predetermined distance until land members 126 of contacting members 125 contact the sclera of eyeball 130. This contact leaves temporary, but visible impressions 134 (i.e., inwardly extending depressions) surrounding an incision site 136 created by trocar 112, for a predetermined time period, such as the length of time needed for a surgical procedure. Accordingly, during a surgical procedure, various instruments may be selectively inserted within incision site 136 quickly and easily as incision site 136 is visible to the surgeon.

It is also understood that in yet another alternative configuration, contacting members 125 may have a bioabsorbable ink or a bioreactive dye applied thereto, similar to what is described above in connection with the embodiment shown in FIGS. 1A-3. Accordingly, when contacting members 125 contact the sclera, the ink or dye serves as a visible indicator to the surgeon.

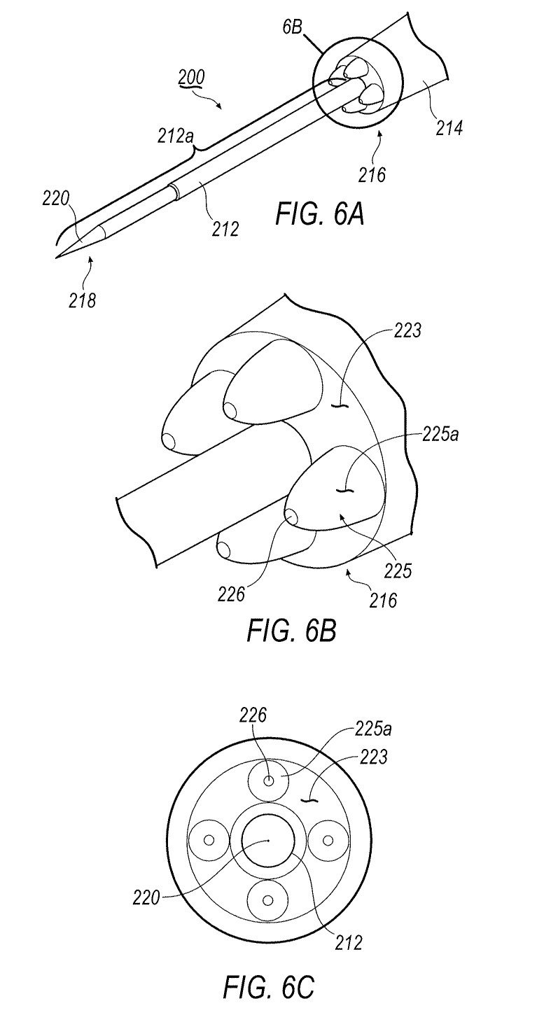

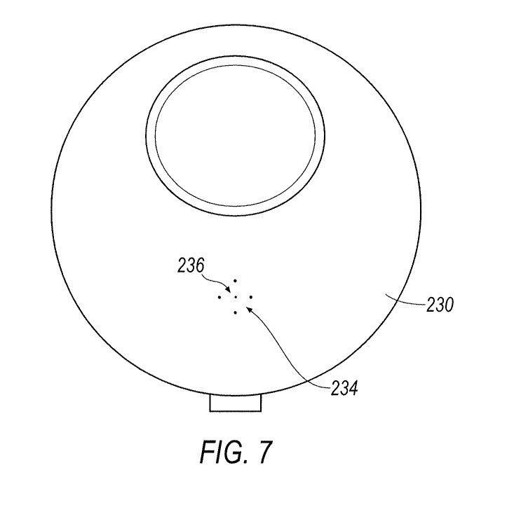

Referring to FIGS. 6A-7, another alternative arrangement of a surgical trocar assembly 200 will now be discussed. Surgical trocar assembly 200 comprises a trocar 212, a handle 214, and a marker element 216. Trocar 212 is generally constructed as an elongated stylet and defined by a distal end 218. Distal end 218 is configured with a tissue piercing tip 220 to facilitate penetration of trocar 212 into a patient's tissue, such as, for example, the eye.

In one exemplary arrangement, handle 214 is configured with a mounting channel (not shown) that receives a proximal portion of trocar 212. The proximal portion of trocar 212 is fixedly secured within the channel. More specifically, the proximal portion of trocar 212 is inserted within handle 214 by a predetermined length, such that a working portion 212a of trocar 212 extends distally away from handle 214 by a predetermined distance that is configured to reach a desired location within a patient.

Marker element 216 is connected to handle 214. In one exemplary configuration, marker element 216 is configured as a generally annular member positioned around trocar 212, and defined by one or more contacting members 225 that extend in a distal direction from a shoulder 223 of handle 214. In one exemplary arrangement, for example, as shown in FIG. 6B, there are four contacting members 225, arranged equi-distance from one another. Each contacting member 225 is defined by a generally conical shaped surface 225a with a distally oriented land member 226 positioned at an apex of contacting member 225. In one exemplary arrangement, each land member 226 has a generally circular shape and may be oriented so as to be transverse to an axis extending through trocar 212. However, it is understood that any number of contacting members 225 may be utilized and that other sizes and shapes of land members 226 may be employed.

Contacting members 225 may be integrally formed with handle 214, as shown in FIG. 6A. Alternatively, marker element 216 may be a separate unit that may be selectively attached to a distal end of handle 214. For example, marker element 216 may be formed with a recess that receives the distal end of handle 214 in a friction fit arrangement when marker element 216 is slid onto trocar 212.

Referring now to FIG. 7, use of surgical trocar assembly 200 will now be explained in the context of a sclerotomy. However, it is understood that surgical trocar assembly 200 may be used in other surgical applications, particularly those which use micro-surgical instruments.

In operation, once a surgical site has been selected, tissue piercing tip 220 is inserted through the sclera of eyeball 230. More specifically, working portion 212a of trocar 212 is inserted into eyeball 230, a predetermined distance until land members 226 of contacting members 225 contact the sclera of eyeball 230. This contact leaves temporary, but visible impressions 234 surrounding an incision site 236 created by trocar 212, for a predetermined time period, such as the length of time needed for a surgical procedure. Accordingly, during a surgical procedure, various instruments may be selectively inserted within incision site 236 quickly and easily as incision site 236 is visible to the surgeon.

It is also understood that in yet another alternative configuration, contacting members 225 may have a bioabsorbable ink or a bioreactive dye applied thereto, similar to what is described above in connection with the embodiment shown in FIGS. 1A-3. Accordingly, when contacting members 225 contact the sclera, the ink or dye serves as a visible indicator to the surgeon.

It will be appreciated that the devices and methods described herein have broad applications. The foregoing embodiments were chosen and described in order to illustrate principles of the methods and apparatuses as well as some practical applications. The preceding description enables others skilled in the art to utilize methods and apparatuses in various embodiments and with various modifications as are suited to the particular use contemplated. In accordance with the provisions of the patent statutes, the principles and modes of operation of this invention have been explained and illustrated in exemplary embodiments.

It is intended that the scope of the present methods and apparatuses be defined by the following claims. However, it must be understood that this invention may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

What is claimed is:

1. A surgical trocar with a marker element, comprising:
a trocar that is constructed as an elongated member defined by distal and proximal ends, wherein the distal end further comprises a tissue piercing tip;
a handle; and
a marker element defining a shoulder, wherein the marker element further comprises at least one contacting member that extends in a distal direction from the shoulder; the contacting member configured to contact a surface of tissue adjacent an incision site created by the tissue piercing tip so as to provide a visual indication of the incision site when the trocar is removed from the incision site.

2. The surgical trocar of claim 1, wherein the marker element is configured as an annular member defined by an internal surface and an external surface that cooperate to form a marker periphery that is arranged around the trocar.

3. The surgical trocar of claim 2, wherein the annular member defines a recess therein, and wherein the trocar is arranged in a central region of the recess.

4. The surgical trocar of claim 3, wherein the annular member is configured to leave a temporary impression on the tissue surrounding the incision site created by the incision site when the marker element is pressed against the tissue.

5. The surgical trocar of claim 2, wherein the annular member is configured with a marking agent such that the annular member contacts the surface of the tissue adjacent an incision site, so as to deposit a visible ring around the incision site created by the trocar.

6. The surgical trocar of claim 5, wherein the marking agent is one of a bioabsorbable ink, bioreactive dye, and bioreactive stain.

7. The surgical trocar of claim 6, wherein the marking agent forms a temporary ring that disappears after a predetermined time period.

8. The surgical trocar of claim 2, wherein the internal surface and external surface are configured to form a taper.

9. The surgical trocar of claim 1, wherein each of the contacting members is defined by opposing surfaces and a distally oriented land member positioned therebetween.

10. The surgical trocar of claim 9, wherein each of the opposing surfaces are contoured inwardly in a distal direction to define edges of the land members.

11. The surgical trocar of claim 9, wherein the marker element includes at least four contacting members, positioned equi-distance from one another around the trocar.

12. The surgical trocar of claim 9, wherein the contacting members are integrally formed with the handle.

13. The surgical trocar of claim 9, wherein the contacting members are configured to leave a temporary impression on the tissue surrounding the incision site created by trocar when the marker element is pressed against the tissue.

14. The surgical trocar of claim 1, wherein each of contacting members is defined by a generally conical shaped member that includes a distally oriented land member positioned at an apex of the contacting member.

15. The surgical trocar of claim 14, wherein the land members have a generally circular shape.

16. The surgical trocar of claim 14, wherein the marker element includes at least four contacting members positioned equi-distance from one another around the trocar.

17. The surgical trocar of claim 14, wherein the contacting members are integrally formed with the handle.

18. The surgical trocar of claim 14, wherein the contact members are configured to leave a temporary impression on the tissue surrounding the incision site created by trocar when the marker element is pressed against the tissue.

19. A marker element for use with a surgical trocar, the marker element defining a shoulder from which at least one contacting member that extends in a distal direction from the shoulder, the contacting member configured to contact a surface of tissue adjacent an incision site created by the tissue piercing tip so as to provide a visual indication of the incision site when the trocar is removed from the incision site.

20. The marker element of claim 19, wherein the marker element is configured as an annular member defined by an internal surface and an external surface that cooperate to form a generally circular marker element that is arranged concentrically around the trocar when the marker element is attached to the trocar.

21. The marker element of claim 20, wherein the annular member is configured with a marking agent such that the annular member contacts the surface of the tissue adjacent an incision site, so as to deposit a visible ring around the incision site created by the trocar.

22. The marker element of claim 20, wherein each of the contacting members is defined by opposing surfaces and a distally oriented land member positioned therebetween, the land members being configured for contacting the surface of the tissue adjacent an incision site, so as to form an impression adjacent the incision site created by the trocar.

23. The marker element of claim 20, wherein each of contacting members is defined by a generally conical shaped member that includes a distally oriented land member positioned at an apex of the contacting member, the land member being configured for contacting the surface of the tissue adjacent an incision site, so as to form an impression adjacent the incision site created by the trocar.
